(19) United States
(12) Patent Application Publication
(10) Pub. No.: US 2023/0301745 A1
(43) Pub. Date: Sep. 28, 2023

(54) SURGICAL ASSEMBLY WITH A LOCKING MECHANISM AND CLAMP DEVICE

(71) Applicant: KOROS U.S.A., INC., Moorpark, CA (US)

(72) Inventors: TIBOR KOROS, Westlake Village, CA (US); AARON STARK, Moorpark, CA (US)

(21) Appl. No.: 18/125,627

(22) Filed: Mar. 23, 2023

Related U.S. Application Data
(60) Provisional application No. 63/322,927, filed on Mar. 23, 2022.

(51) Int. Cl. A61B 90/57; A61B 17/02

(52) U.S. Cl. CPC A61B 90/57; A61B 17/02

(57) ABSTRACT

Disclosed herein are surgical assemblies comprising clamps with a lever clamp and a retractor arm that utilizes a ball-locking mechanism for clamping medical retractors. The ball-locking mechanism allows the retractor to be rotatable relative to the rod of the retractor arm. These assemblies allow for streamlined, accurate, efficient, and quick adjustments to retractors prior to or during surgery. They also obviate the need for a medical professional to hold the retractor in place during surgery and could reduce risk of human error during surgeries.

### RELATED APPLICATION

This application claims the priority benefit of U.S. Provisional Patent Application No. 63/322,927 to Koros et al., entitled “Surgical Assembly with a Locking Mechanism and Clamp Device” and filed on Mar. 23, 2022, the entirety of which is incorporated by reference herein.

### BACKGROUND

### Field of the Disclosure

The present disclosure is related generally to improvements to mechanisms, devices, and systems for use in the medical field for surgical procedures, and specifically, to a locking mechanism for a retractor arm and a clamp device that may be used together in an assembly with retractors to replace or reduce the number of medical professionals assisting during surgical procedures.

### Description of the Related Art

The use of clamps, retractors, and retractor arms together during surgery is common in the medical field. These devices may be used separately or concurrently as a system. Retractors help operating room professionals hold an incision or wound open during surgical procedures. They may also help with holding back underlying organs or tissue to allow a doctor or nurse better visibility and access during surgery.

Retractors may be hand-held or may be held by another rod, often cylindrical, with a clamp on at least one end. These devices are often referred to as retractor arms. The clamps at the end of a retractor arm are typically static when the clamp is tightened, so if an operating room professional wants to adjust the angle or positioning of the retractor, he or she must do so manually, meaning he or she must loosen the clamp and before adjusting the retractor and then retightening the clamp. These adjustments take time and energy of a medical professional, and, in certain situations, time and energy during surgery plays a critical part in ensuring a successful patient outcome. Thus, there is a need for a system of retractor arms, retractors, and clamps that allow easy and/or quick adjustments in angle or positioning of the attached retractor.

There are also clamps that medical professionals use to hold one or more retractor arms in place during surgery. These clamps may be engaged or disengaged using a lever mechanism. Typically, these levers are short and angled downward. However, if a surgeon wishes to use thicker, longer retractor arms during surgery (for example orthopedic surgery, which typically requires larger, stronger devices), a large amount of strength or torque is required to engage the clamp and lock the thicker retractor arm in place. The amount of torque necessary to lock a retractor arm with a thicker diameter is difficult to generate with prior art levers. And, the angled nature of prior art lever handles often times will lead to interference with longer retractor arms. Thus, there is a need for a clamp with a lever that allows for easier engagement or disengagement and does not interfere with the retractor arms being clamped in place, or even other medical devices being utilized around the surgical table.

Finally, hand retractors may be used by surgeons as a device during surgery to, among other things, provide a better view of certain interior portions of the surgical subject. While hand retractors are useful, they occupy a medical professional's hand during operation. As such, there is a need for a clamp that frees the medical professional's hands for operation, and/or that reduces the number of medical personal needed for an operation, which creates more working space in the surgical room and reduces the risk of human error.

### SUMMARY OF THE DISCLOSURE

One embodiment of a surgical retractor arm with ball-locking mechanism according to the present disclosure comprises a rod, a lock-and-release mechanism connected to the rod, and a ball component, with the outer surface of the ball component configured to engage with the lock-and-release mechanism. Embodiments of the retractor arm according to the present disclosure further includes a retractor clamp connected to the ball component, and a clamp screw that connects a clamp driver to the retractor clamp. The clamp driver is configured to control the clamp screw, and the clamp screw is in turn configured to engage the retractor clamp.

One example of a clamp device according to the present disclosure comprises at least two hollow components, a flat lever connected to the hollow components, and a locking mechanism that is integrated with the hollow components and the flat lever. The locking mechanism is controlled by the flat lever and allows for the tightening or loosening of the hollow components.

One example of a surgical assembly according to the present disclosure comprises at least one surgical table clamp configured to securely connect to a surgical table. The assembly further comprises a first rod connected to the surgical table clamp, a first clamp device connected to the rod, and a first retractor arm connected to the first clamp device. The rod or rods may be straight or angled/bent (e.g., in an L-shape).

These and other further features and advantages of the invention would be apparent to those skilled in the art from the following detailed description, taken together with the accompanying drawings, wherein like numerals designate corresponding parts in the figures, in which:

### BRIEF DESCRIPTION OF THE DRAWINGS

FIG. 1 shows a front perspective view of one embodiment of a surgical assembly according to the present disclosure.

FIGS. 2A and 2B show top and side views, respectively, of a ball-locking mechanism in a retractor arm device that was shown integrated into the assembly of FIG. 1, in a detached configuration.

FIG. 3A shows a side view of the ball-locking mechanism and retractor clamp with clamp driver in a retractor arm device shown in FIGS. 2A and 2B in an attached configuration, and FIG. 3B shows a magnified view of a portion thereof.

FIG. 4A shows a cross-sectional view of the ball-locking mechanism and retractor clamp with clamp driver in a retractor arm device shown in FIGS. 2A and 2B, and FIG. 4B shows a magnified view of a portion thereof.

FIG. 5 shows an exploded perspective view of the ball-locking mechanism in a retractor arm device shown in FIGS. 2A and 2B.

FIGS. 6-9 show side, top perspective, bottom perspective, and top views, respectively, of a clamp device that was shown integrated into the assembly of FIG. 1.

FIGS. 10-12 show top perspective, side, and bottom views, respectively, of the clamp device shown in FIGS. 6-9, in a second configuration.

FIGS. 13-17 show various views of an embodiment of the clamp device and the ball-locking mechanism in a retractor arm device that was shown integrated into the assembly of FIG. 1.

FIG. 18 shows a top view of one embodiment of the ball-locking mechanism in a retractor arm device of FIGS. 1-5 with a retractor held in place by the retractor arm's clamp at one end.

FIGS. 19-23 show various views of another embodiment of the clamp device and the ball-locking mechanism in a retractor arm device that was shown integrated into the assembly of FIG. 1.

FIG. 24 shows a top view of and the ball-locking mechanism in a retractor arm device of FIGS. 1 and 19-23 with a retractor held in place by the retractor arm's clamp at one end.

### DETAILED DESCRIPTION OF THE DISCLOSURE

Embodiments incorporating features of the present disclosure include surgical assemblies comprising clamps with a lever clamp (e.g., a flat lever clamp) and a retractor arm that utilizes a ball-locking mechanism for clamping medical retractors. The ball-locking mechanism allows the retractor to be rotatable relative to the rod of the retractor arm. These assemblies allow for streamlined, accurate, efficient, and quick adjustments to retractors prior to or during surgery. They also obviate the need for a medical professional to hold the retractor in place during surgery and could reduce risk of human error during surgery.

In the following detailed description, numerous specific details are set forth in order to provide a more thorough understanding of embodiments incorporating features of the present disclosure. However, it will be apparent to one skilled in the art that devices, methods, and assemblies according to the present disclosure can be practiced without necessarily being limited to these specifically recited details.

Embodiments of the disclosure are described herein with reference to illustrations that are schematic illustrations of embodiments of the disclosure. As such, the actual size, components and features can be different, and variations from the shapes of the illustrations as a result, for example, of technological capabilities, manufacturing techniques and/or tolerances are expected. Embodiments of the disclosure should not be construed as limited to the particular shapes or components of the regions illustrated herein but are to include deviations in shapes/components that result, for example, from manufacturing or technological availability. The regions illustrated in the figures are schematic in nature and their shapes are not intended to illustrate the precise shape or functionality of a feature of a device and are not intended to limit the scope of the disclosure. In addition, components may be shown as one unit but may instead be a collection of components or units, or a collection of components or units may exist as one unit.

Throughout this description, the preferred embodiment and examples illustrated should be considered as exemplars, rather than as limitations on the present disclosure. As used herein, the term “disclosure,” “device,” “assembly,” “system” “method,” “present disclosure,” “present device” or “present method” refers to any one of the embodiments of the disclosure described herein, and any equivalents. Furthermore, reference to various feature(s) of the “disclosure,” “device,” “assembly,” “system,” “method,” “present disclosure,” “present device” or “present method” throughout this document does not mean that all claimed embodiments or methods must include the referenced feature(s).

It is also understood that when an element or feature is referred to as being “on” or “adjacent” another element or feature, it can be directly on or adjacent to the other element or feature, or intervening elements or features may also be present. It is also understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present.

Relative terms such as “outer,” “above,” “lower,” “below,” “horizontal,” “vertical” and similar terms, may be used herein to describe a relationship of one feature to another. It is understood that these terms are intended to encompass different orientations in addition to the orientation depicted in the figures.

Although the terms first, second, etc. may be used herein to describe various elements or components, these elements or components should not be limited by these terms. These terms are only used to distinguish one element or component from another element or component. Thus, a first element or component discussed below could be termed a second element or component without departing from the teachings of the present disclosure. As used herein, the term “and/or” includes any and all combinations of one or more of the associated list items.

The terminology used herein is for describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

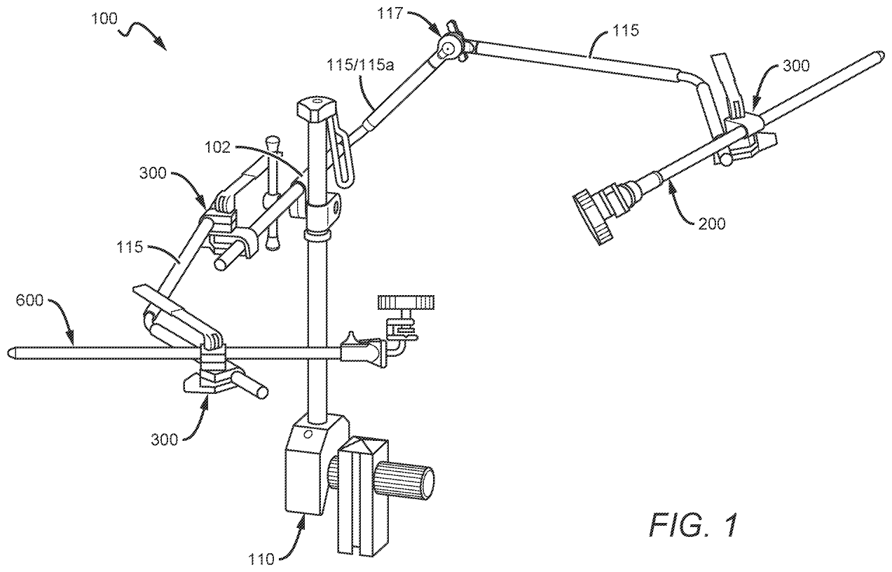

FIG. 1 shows an embodiment of a surgical assembly 100 incorporating features of the present disclosure. The embodiment of the surgical assembly 100 shown in FIG. 1 comprises a surgical table clamp 110, two angled or bent cylindrical rods 115, three clamps 300 (referred to herein as “flat clamps” though it is understood that other types of clamps are possible), and two embodiments of retractor arms 200, 600 each with a ball-locking mechanism. It is understood that less or additional numbers of each of the components are possible and that many different embodiments and variations are possible allowing for additional retractors to assist with surgery or different positioning of the retractors in relation to the surgical table. It is also understood that the rods may be square rather than cylindrical, or some other shape, and that the corresponding apertures described herein can be shaped so as to match the shape of the rods.

The components that make up the surgical assembly 100 connect to one another. For example, in the embodiment shown in FIG. 1, the surgical table clamp 110, among other things, comprises a medical clamp 102 with a cylindrically hollow piece where one side of an angled rod 115a can pass through and be locked into place to the surgical table clamp 110. The hollow piece of the medical clamp 102 may have other shapes that match the shape of the rod that is to be passed through. The cylindrically hollow piece in the medical clamp 102 can be part of the clamp, or can be a separate component attached thereto. In the embodiment shown, the cylindrically hollow piece is configured such that when a rod is passed through, it is substantially horizontal (parallel relative to the floor) and substantially perpendicular to the surgical table clamp 110, though other embodiments are possible. In some embodiments the angled rods 115 may be bent, for instance, in approximately the middle of the rod at approximately at a 90 degree angle, though other angles are possible (e.g., between 30 and 120 degrees). While in the embodiment shown each of the angled rods 115 has multiple thicknesses or diameters, in some embodiments the angled rod 115 may have a uniform thickness or diameter throughout the entire component. It is understood that other embodiments are possible. The angled rods 115 may be bent or angled in multiple places throughout the rods. It is understood that other mechanisms or devices for locking/unlocking the angled rods 115 are possible. It is also possible, in some embodiments, for the rods to be straight or bent at different angles. The cylindrical rods 115 may also be configured with a mechanism 117 that allows a portion of it to rotate, pivot, or otherwise form an angle about the mechanism. Moreover, not all the rods in the surgical assemblies disclosed herein need to be identical; some rods may be straight, and some may be bent, and some rods may have different diameters from one another.

The opposite ends of the angled rod 115a can be passed through the surgical table clamp 110 via the medical clamp 102 with a cylindrically hollow piece and locked to one or more flat clamp devices 300. This means it is possible for at least two flat clamps 300 to be attached to the bent cylindrical rod 115a (which is itself attached to the surgical table clamp 110): one flat clamp device 300 on the right side of the angled rod 115a and a second flat clamp device 300 on the left side of the angled rod 115a, as shown in FIG. 1.

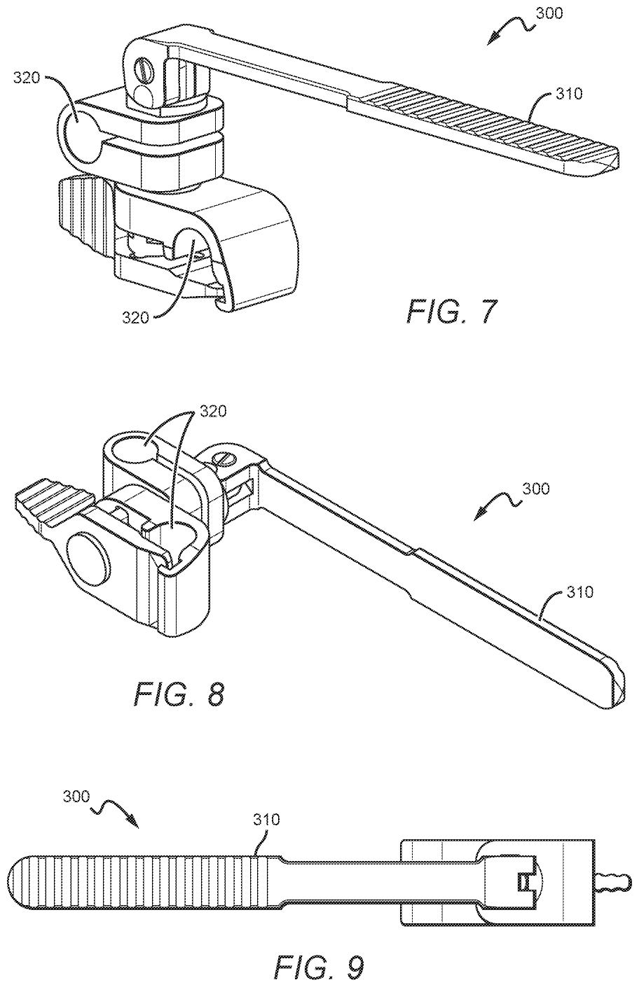

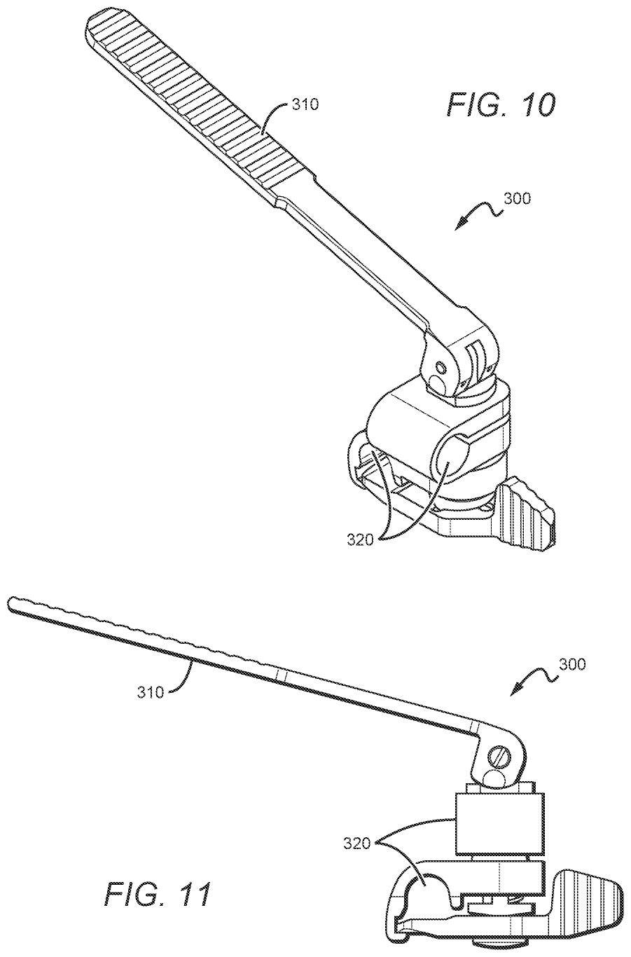

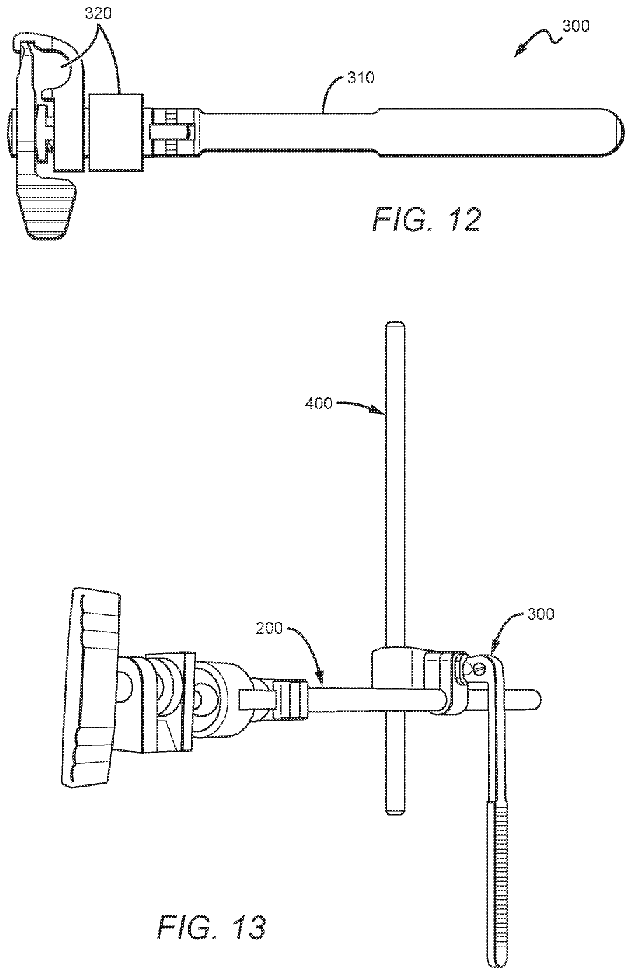

One embodiment of a flat clamp device 300 according to the present disclosure can comprise two cylindrically hollow components, a flat lever, and a tightening or locking mechanism that connects the two cylindrically hollow components and the flat lever. One embodiment of the flat clamp device is shown in more detail in FIGS. 6-12 and will be discussed in detail below.

Referring again to FIG. 1, with respect to each of the flat clamps 300 connected to the angled cylindrical rod 115a, another angled cylindrical rod 115 may be passed through and locked in the other hole of each of the two flat clamps 300 attached to the surgical table clamp 110 by angled cylindrical rod 115a. The other ends of those additional angled cylindrical rods 115 may be passed through and locked into additional (e.g., fourth, fifth, etc.) flat clamps 300, or other devices such as retractor arm 200. In this embodiment of the surgical assembly 100, this results in two flat clamps 300 on either side of the surgical table clamp. The retractor arms 200 and the cylindrical rods 115,115a may be of the same, uniform thickness or of varying thicknesses.

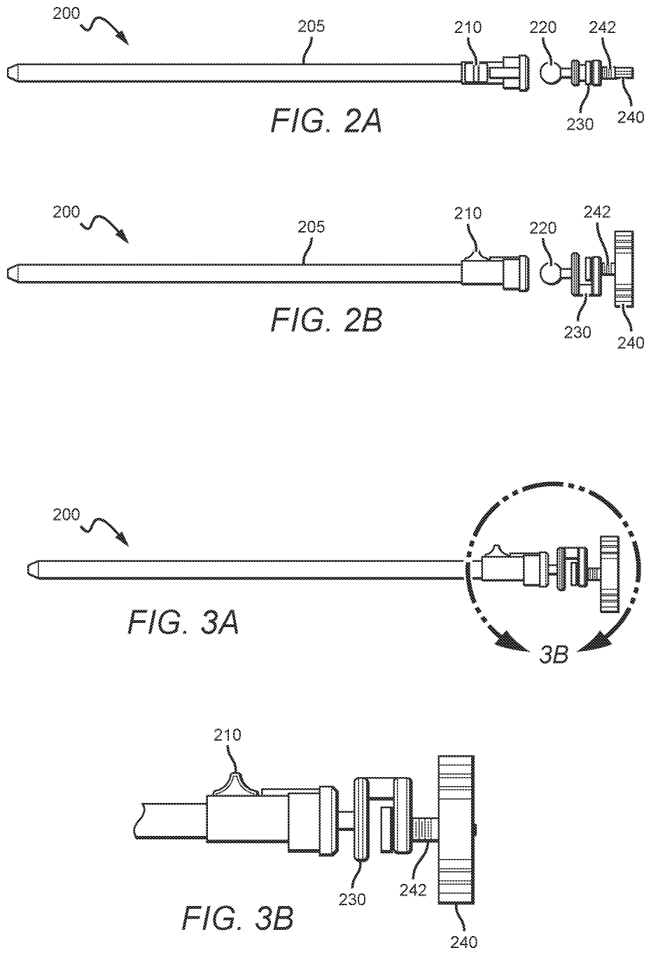

FIGS. 2-5 show various views of an embodiment of the retractor arm 200 with a ball-locking mechanism (which can comprise the ball component 220 and the lock-and-release mechanism 210), comprising a shaft or cylindrical rod 205 (though other shapes are possible), a lock-and-release mechanism 210, a ball component 220 that fits in the lock-and-release mechanism 210, a retractor clamp 230, a clamp driver 240, and a clamp screw 242. Although the ball component 220 is referred herein as being in the shape of a ball (i.e., spherical), it is understood that other shapes and embodiments are possible to achieve the goal of a locking mechanism. In some embodiments the ball component 220 and the retractor clamp 230 can be a monolithic piece and/or integral with one another, though other embodiments are possible.

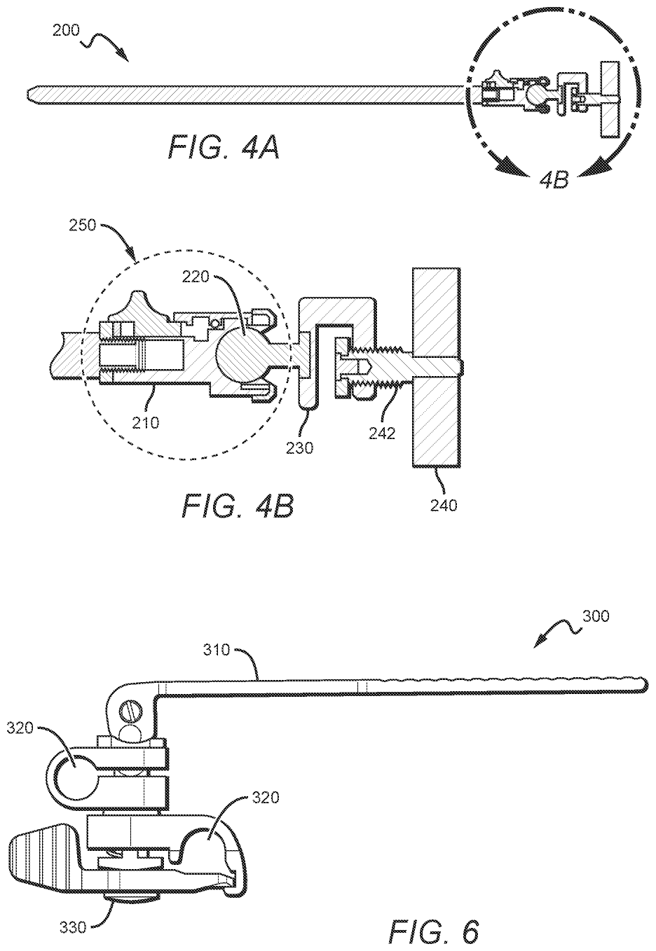

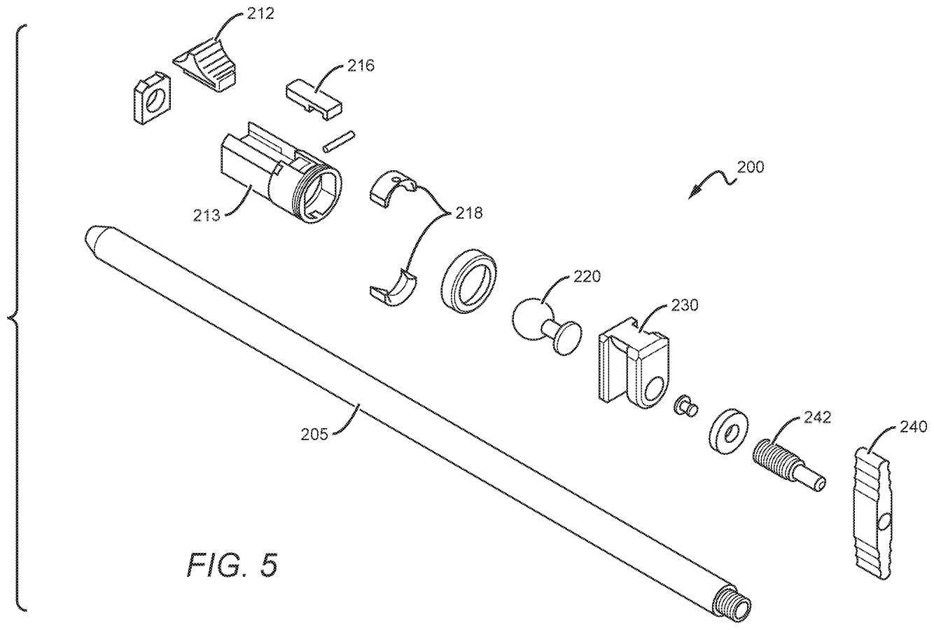

In some embodiments, as can be seen in the exploded view of FIG. 5, the lock-and-release mechanism 210 comprises a spring lock 216, a sliding trigger 212 used to engage and disengage the spring lock 216, a housing 213 for the mechanism 210, and a ball lock 218. When the spring lock 216 is engaged, the ball lock 218 is engaged. Disengaging the sliding trigger 212 by sliding it (e.g. sliding it vertically downwards along its length away from the retractor clamp 230) in turn disengages and unlocks the spring lock 216, which then unlocks the ball lock 218 and allows the pieces of the ball lock 218 to become separable within the housing 213. When the sliding trigger 212 and the spring lock 216 are engaged, the pieces of the ball lock 218 are locked together, and the ball component 220 is locked in place relative to the rod 205, as shown in FIG. 4B.

In other embodiments, the lock-and-release mechanism 210 comprises a plunger lock, a sliding trigger used to engage and disengage the plunger lock, a housing for the mechanism, and a ball lock. When the plunger lock is engaged, the ball lock is engaged. Disengaging the sliding trigger by sliding it (e.g., sliding it vertically downwards along its length away from the retractor clamp 230) in turn disengages and unlocks the plunger lock and allows the pieces of the ball lock to become separated within the housing. In this embodiment, when the sliding trigger is disengaged, it remains in the disengaged position without any pressure from a user. This allows for easy one-handed use of the lock-and-release mechanism 210 and, ultimately, the retractor arm. When the sliding trigger and the plunger lock are engaged, the pieces of the ball lock are locked together, and the ball component is locked in place relative to the rod. It should be understood that other lock-and-release mechanisms are possible (e.g., a pin-lock system).

The housing 213, ball lock 218, and ball component 220 can be connected to each other like a ball-and-socket joint. The housing 213 can include a socket such that the outer surface of the ball component 220 engages with the inner surface of the side of the housing 213 that interacts with the ball lock 218 such that the ball component 220 is rotatable within the housing 213, even when the ball lock is closed or engaged. But, when closed, locked, or engaged, the ball component 220 in the housing 213, the ball lock 218 prevents the ball component 220 from being removed from the well of the housing 213, and the ball component 220 is only rotatable.

The inner diameter of the ball lock 218 is smaller than the outer diameter of the ball component 220 to engage the ball component 220 and lock the ball component 220 so that it is only rotatable within the housing. The inner diameter of the ball lock 218 is bigger than the outer diameter of the ball component 220 when the ball lock 218 is unlocking or disengaging with the ball component 220, so as to allow for removal of the ball component 220 from the ball lock 218. In some embodiments, the ball component 220 can have a diameter of approximately 0.25 inches or more, 0.5 inches or more, 1 inches or more, or 0.25 to 1 inches.

FIGS. 6-12 show various views of an embodiment of the flat clamp device 300, comprising two cylindrically hollow components 320, a flat lever 310, and a tightening or locking mechanism that connects the two cylindrically hollow components 320 and the flat lever 310. While the term “flat” is used herein to describe various elements (e.g., the flat clamp device 300 and the flat lever 310), it is understood that non-flat embodiments are also possible. Moreover, while the term “cylindrically hollow” is used herein to describe various elements such as the components 320, it is understood that the phrase “cylindrically hollow” encompasses components that are partially cylindrically hollow (e.g., the lower component 320 in FIG. 6), and that shapes other than cylindrical are possible.

The flat lever 310 controls the locking mechanism of the flat clamp device 300 by controlling whether the cylindrically hollow components 320 are in a locked or unlocked position. For example, when the flat lever 310 is moved toward the tightened position, the cylindrically hollow components 320 become smaller (e.g., tighter or smaller in diameter). In some embodiments, the locking mechanism works with the flat lever 300 to vertically loosen or compress the cylindrically hollow components 320. The flat lever 310 shown in FIGS. 6-12 controls the cylindrically hollow components 320 simultaneously, but it is possible for the flat lever 310 to control the components separately or at different rates of compression/decompression. In some embodiments, the flat lever 310 can have a length of 5 inches or more, 6 inches or more, 7 inches or more, 8 inches or more, 8 inches or more, 10 inches or more, 12 inches or more, 5 to 20 inches, or approximately 8 inches. It is understood that these ranges are exemplary in nature and lengths outside these ranges are possible.

The cylindrically hollow components 320 shown in FIGS. 6-12 are immovably attached to one another. However, it is understood that the components 320 may be rotatable to one another to allow for different, customizable configurations of the assembly 100.

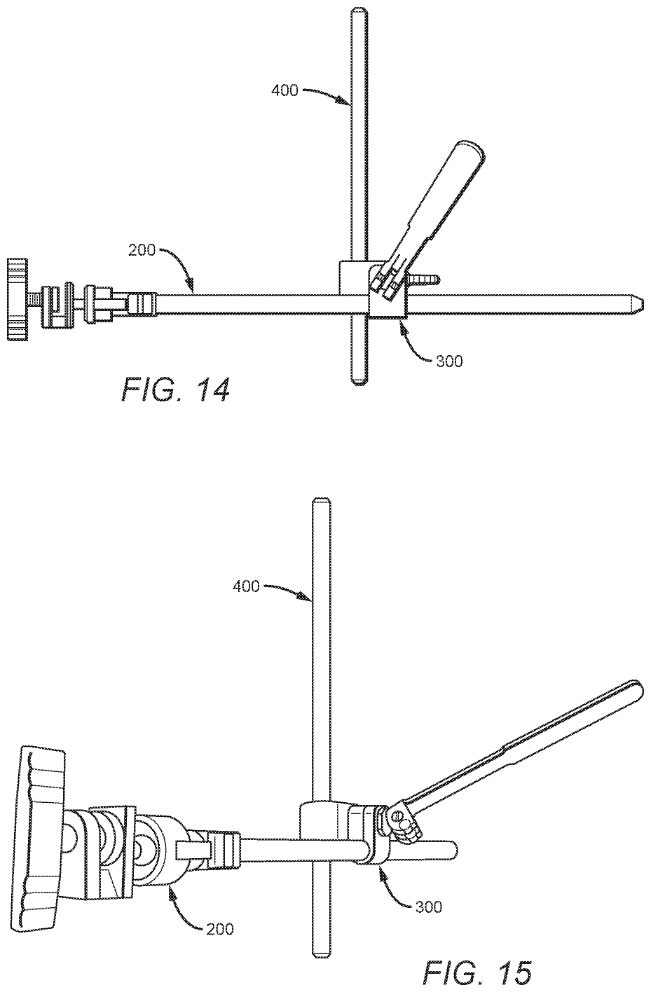

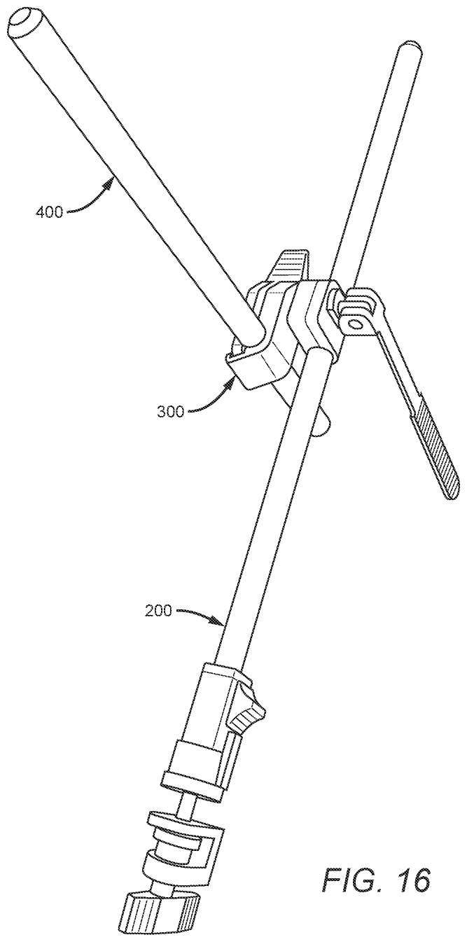

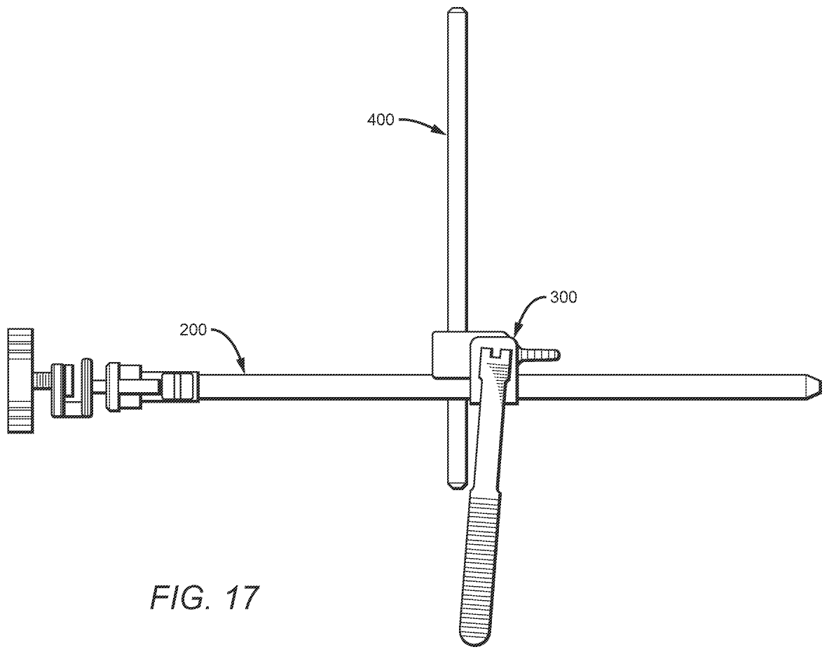

FIGS. 13-17 show various views of one embodiment of one aspect of the assembly comprising a flat clamp device 300, a retractor arm 200 with ball-locking mechanism, and a straight rod 400. FIGS. 13-17 highlight the customizability of assemblies 100. FIGS. 13, 16, and 17 show the flat clamp device 300 fully engaged (the lever is pushed down to its locking point) and the retractor arm 200 with ball-locking mechanism and the rod 400 are locked in place. FIGS. 14 and 15 show the flat clamp device 300 fully disengaged.

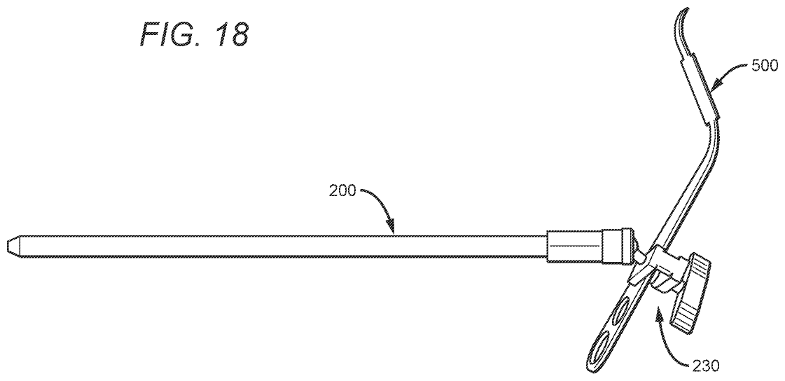

FIG. 18 shows a top plan view of one embodiment of the retractor arm device 200 with ball-locking mechanism with a retractor 500 locked in place using the retractor arm's 200 retractor clamp 230, the clamp driver 240, and clamp screw 242. The retractor 500 is locked in place relative to the retractor clamp 230, the clamp driver 240, and clamp screw 242, but is rotatable at least 180 degrees relative to the rod 205 due to the ball joint 220 in the ball-locking mechanism. Embodiments according to the present disclosure can result in a retractor 500 being held by the retractor clamp 230, the clamp driver 240, and clamp screw 242, with the ability to expose various sections of the body during surgery and keep them exposed without being held by a medical professional. The retractor 500 is also easily and quickly adjustable, in large part due to the retractor arm's 200 ball-locking mechanism.

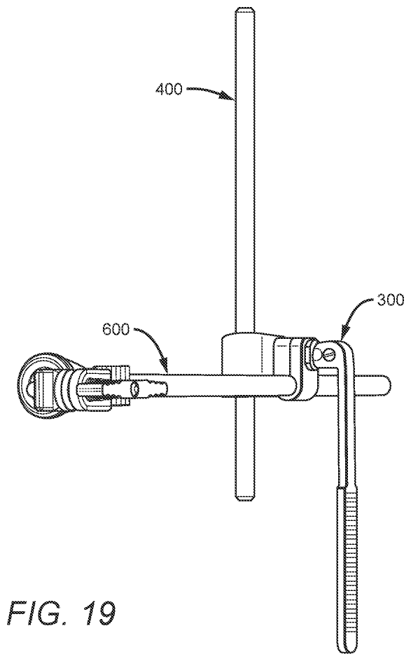

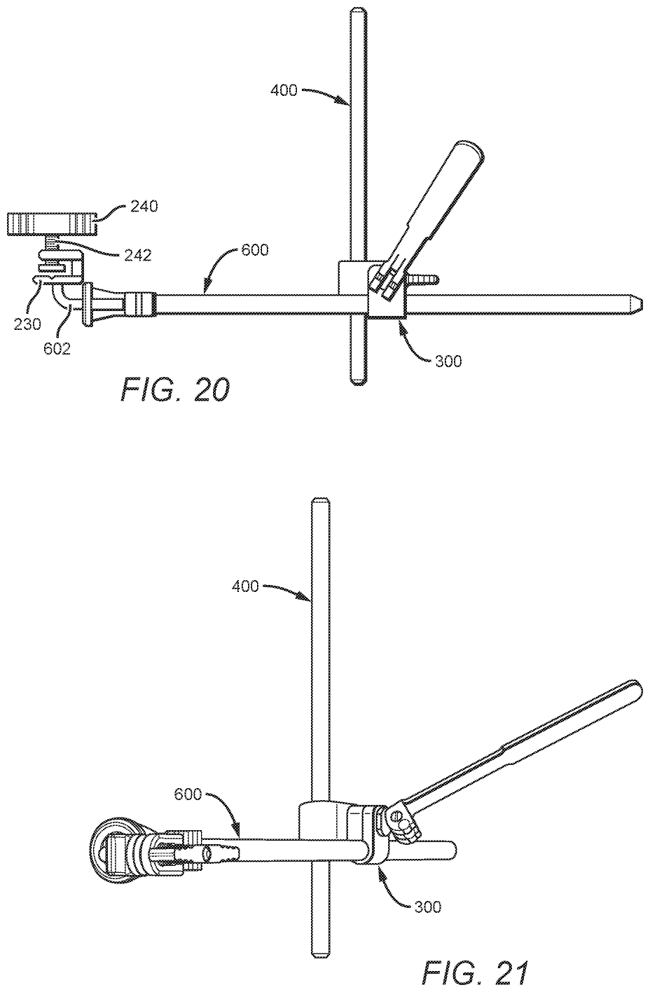

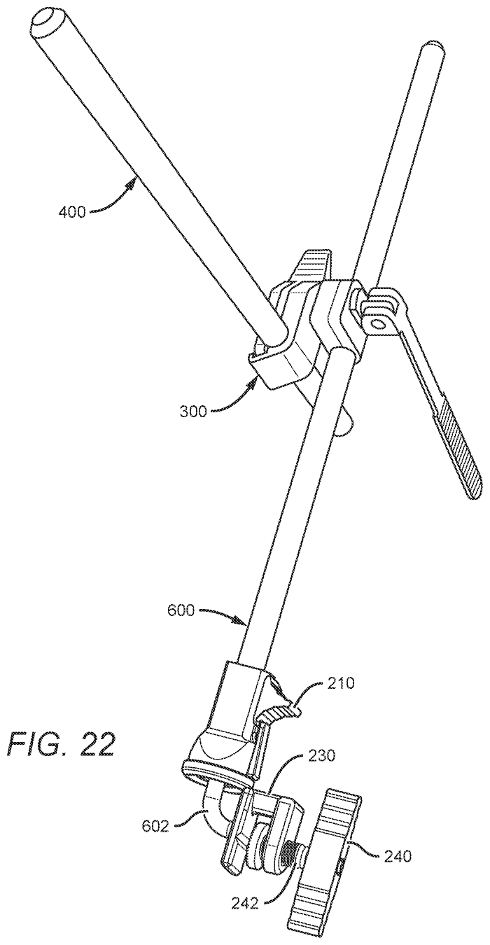

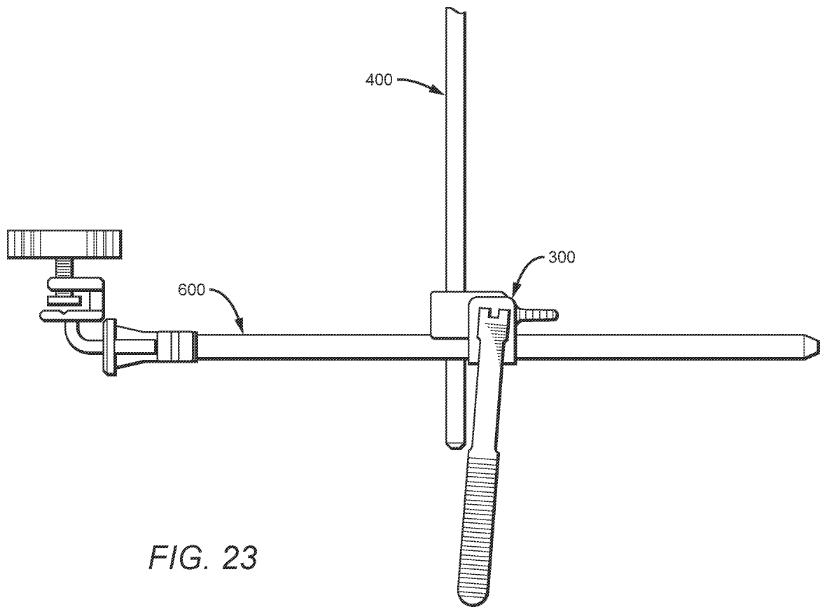

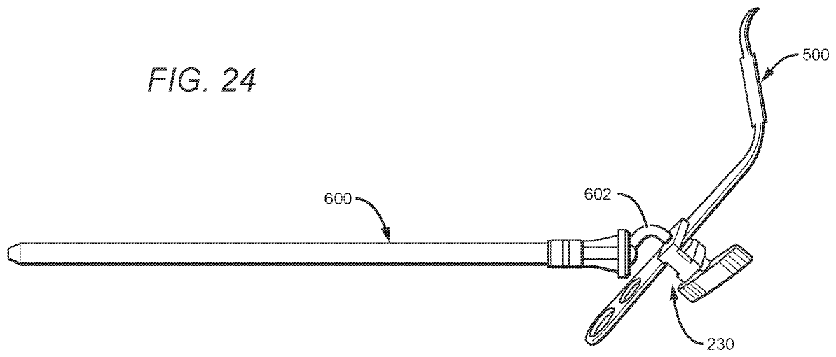

FIGS. 19-23 show various views of another embodiment of one aspect of the assembly comprising a flat clamp device 300, a retractor arm 600 with ball-locking mechanism, and a straight rod 400. The difference between the embodiment shown in FIGS. 13-17 and the embodiment shown in FIGS. 19-23 is the retractor arm 600 in FIGS. 19-23 is configured such that the retractor clamp 230, the clamp driver 240, and clamp screw 242 are collectively angled relative to the ball joint 220. FIGS. 20 and 22 best show this retractor arm 600 with ball-locking mechanism embodiment bent at a point 602. The angle may range from zero to 90 degrees (though other angles are possible), but in the embodiment shown in FIGS. 19-23, the angle is approximately 90 degrees. And, FIG. 24 shows a top plan view of the embodiment of the retractor arm device 600 with ball-locking mechanism shown in FIGS. 19-23, with a retractor 500 locked in place using the clamp retractor clamp 230, clamp driver 240, and clamp screw 242 of the retractor arm 600. It is understood that an embodiment of a retractor arm 600 with ball-locking mechanism with a bent point 602 may have many different types of lock-and-release mechanisms such as a spring lock or a plunger lock.

It is understood that embodiments presented herein are meant to be exemplary. Although the present disclosure has been described in detail with reference to certain preferred configurations thereof both in the specification and in the claims, other versions are possible. Embodiments of the present disclosure can comprise any combination of compatible devices/features described herein and/or shown in the figures, and these embodiments should not be limited to those expressly illustrated and discussed. For instance and not by way of limitation, the appended claims could be modified to be multiple dependent claims so as to combine any combinable combination of elements within a claim set, or from differing claim sets. Claims depending on one independent claim could be modified so as to depend from a different independent claim. Therefore, the spirit and scope of the disclosure should not be limited to the versions described above.

While the foregoing written description of the disclosure enables one of ordinary skill to make and use what is considered presently to be the best mode thereof, those of ordinary skill will understand and appreciate the existence of variations, combinations, and equivalents of the specific embodiments, methods, systems, and examples herein. The disclosure should therefore not be limited by the above described embodiments, methods, systems, and examples. Furthermore, certain terminology has been used for the purposes of descriptive clarity, and not to limit the present disclosure. It is therefore intended that the following appended claims include all such alterations, modifications and permutations as fall within the true spirit and scope of the present disclosure. No portion of the disclosure is intended, expressly or implicitly, to be dedicated to the public domain if not set forth in the claims.

We claim:

1. A surgical retractor arm with a ball-locking mechanism, comprising:
a rod;
a lock-and-release mechanism connected to said cylindrical rod;
a ball component, wherein the outer surface of said ball component is configured to engage with said lock-and-release mechanism;
a retractor clamp connected to said ball component; and
a clamp driver connected to said retractor clamp by a clamp screw.

2. The surgical retractor arm with a ball-locking mechanism of claim 1, wherein said rod is cylindrical.

3. The surgical retractor arm with a ball-locking mechanism of claim 1, wherein said lock-and-release mechanism comprises:
a ball lock shaped to lock and release said ball component;
a spring lock configured to engage and disengage said ball lock;
a sliding trigger configured to engage and disengage said spring lock; and
a housing, wherein said ball lock, said spring lock, and said sliding trigger are at least partially in said housing.

4. The surgical retractor arm with a ball-locking mechanism of claim 3, wherein said ball component is rotatable within said housing when said ball component is locked by said ball lock.

5. The surgical retractor arm with a ball-locking mechanism of claim 3, wherein said ball lock comprises two or more pieces that when connected define a diameter smaller than the diameter of said ball component, and when unlocked define a diameter larger than the diameter of said ball component.

6. The surgical retractor arm with a ball locking mechanism of claim 1, wherein said lock-and-release mechanism comprises:
a ball lock shaped to lock and release said ball component;
a plunger lock configured to engage and disengage said ball lock;
a sliding trigger configured to engage and disengage said plunger lock; and
a housing, wherein said ball lock, said plunger lock, and said sliding trigger are at least partially in said housing.

7. The surgical retractor arm with a ball locking mechanism of claim 1, wherein said retractor clamp, clamp driver, and clamp screw are angled relative to said ball component.

8. A clamp device comprising:
at least two hollow components each configured to allow a rod to pass through;
a flat lever connected to said at least two hollow components; and
a locking mechanism integrated with said hollow components and said flat lever, wherein said locking mechanism is configured to tighten and loosen said hollow components using said flat lever.

9. The clamp device of claim 8, wherein said hollow components are cylindrically hollow.

10. The clamp device of claim 8, wherein each of said hollow components is configured to lock a rod in place when said locking mechanism is engaged by said flat lever.

11. The clamp device of claim 8, further comprising at least one rod passing through one of said hollow components, wherein said rod is bent.

12. The clamp device of claim 11, wherein said rod is bent at an angle between 30 and 120 degrees.

13. The clamp device of claim 8, further comprising at least one rod passing through one of said hollow components, wherein said rod has a uniform thickness throughout the entire rod.

14. The clamp device of claim 8, wherein said flat lever has a length of 5 inches or more.

15. A surgical assembly, comprising:
at least one surgical table clamp configured to securely connect to a surgical table;
a first rod connected to said surgical table clamp;
a first clamp device connected to said first cylindrical rod; and
a first retractor arm connected to said first clamp device.

16. The surgical assembly of claim 15, wherein said rod is cylindrical.

17. The surgical assembly of claim 15, wherein said first retractor arm comprises a ball-locking mechanism, said ball-locking mechanism comprising:
a lock-and-release mechanism;
a ball component, wherein the outer surface of said ball component is configured to engage with said lock-and-release mechanism;
a retractor clamp attached to said ball component; and
a clamp driver connected to said retractor clamp by a clamp screw.

18. The surgical assembly of claim 17, wherein said ball component, said retractor clamp, said clamp driver, and said clamp screw are detachable from said lock-and-release mechanism.

19. The surgical assembly of claim 17, further comprising at least one retractor, wherein said retractor is secured to said retractor arm by said retractor clamp, said clamp driver, and said clamp screw.

20. The surgical assembly of claim 15, wherein said retractor is immovably locked relative to said retractor clamp and said clamp driver with clamp screw, but is rotatable relative to said rod of said retractor arm via said ball component.
